(19) United States
(12) Patent Application Publication
(10) Pub. No.: US 2011/0104817 A1
(43) Pub. Date: May 5, 2011

(54) INTEGRATED MICRO DEVICE, A METHOD FOR DETECTING BIOMARKERS USING THE INTEGRATED MICRO DEVICE, A METHOD FOR MANUFACTURING AN INTEGRATED MICRO DEVICE, AND AN INTEGRATED MICRO DEVICE ARRANGEMENT

(75) Inventors: Tae Goo Kang, (US); Ajay Agarwal, (US); Hongmiao Ji, (US); Guojun Zhang, (US); Yu Chen, (US)

(73) Assignee: Science, Technology and Research

(21) Appl. No.: 12/907,191

(22) Filed: Oct. 19, 2010

(30) Foreign Application Priority Data
Oct. 20, 2009 (SG) 200907006-1

(51) Int. Cl. G01N 1/18; H01L 21/98; B01L 3/00; C12M 1/34; G01N 33/48

(52) U.S. Cl. 436/177; 438/49; 422/502; 422/68.1; 435/287.1; 257/E21.705

(57) ABSTRACT

Embodiments provide an integrated micro device. The integrated micro device comprises a substrate, a first microfluidic device disposed over a first surface of the substrate, a second microfluidic device disposed over a second surface of the substrate, and at least one via hole through the substrate connecting the first microfluidic device and the second microfluidic device. The second surface of the substrate is opposite to the first surface of the substrate. The first microfluidic device is monolithically integrated with the substrate, and the second microfluidic device is monolithically integrated with the substrate.

The present application claims the benefit of the Singapore provisional application 200907006-1 (filed on 20 Oct. 2009), the entire contents of which are incorporated herein by reference for all purposes.

### TECHNICAL FIELD

Embodiments relates generally to an integrated micro device, a method for detecting biomarkers using the integrated micro device, a method for manufacturing an integrated micro device, and an integrated micro device arrangement.

### BACKGROUND

Cardiovascular disease, such as myocardial infarction (heart attack), are the major cause of death among adults worldwide. A heart attack takes place when the heart muscle is damaged and unable to fulfill its pumping role to distribute blood and oxygen throughout the body. Today when a patient presents himself to the Emergency Department (ED) with a symptom that is suspicious of heart disease, e.g. chest pain, the first assessment is generally to perform an electrocardiogram (ECG) to examine if the heart beats in unison. However ECG lacks sensitivity and fails to detect all myocardial injuries. An injury to the heart muscle is usually synonymous with the death of cardiac cells. With the absence of the cardiac cells, the specific protein biomarkers will be released and trace their way to the blood circulation. Examples of the proteins that may be released due to absence of cardiac cells include, for example, troponin T (cTnT), creatine kinase MM (CK-MM), and creatine kinase MB (CK-MB). Early detection of these protein biomarkers in the circulation has been recognized as the first and foremost defense line against this silent deadly disease. A biomarker generally refers to a substance used as an indicator of a biological state. For example, cTnT, CK-MM, or CK-MB may be used as biomarkers as the indicator of an injury to the heart muscle. Besides proteins, other biomolecules may also be biomarkers as an indicator of a respective biological state. Such biomolecules include, for example, a nucleic acid (such as DNA and RNA), a polypeptide, a small organic molecule and an inorganic molecule etc.

Usually a diagnostic kit for the detection of biomarkers includes a sample preparation module and a diagnostic module. For example, the sample preparation module may be a plasma separation device which separates plasma from a blood sample for further detection of biomarkers, e.g. cTnT, CK-MM, or CK-MB. Standard assays like enzyme-linked immunosorbent assay (ELISA) can detect fairly low traces of cardiac proteins (with the limit of detection having a level of details>10 pg/mL), but they need to be performed centralized in clinical laboratories and hence take time to deliver results (e.g. more than 6 hours). For the realization of point-of-care (POC) diagnostics kits, it is desirable to conduct the diagnosis using only a few drops of blood in a finger prick with faster processing time, e.g. within 30 minutes. Normally the separated plasma flow rate through the outlet of a plasma separation micro device, such as micro filter chip, is quite low, as described in T. G. Kang, et al, “A Continuous Flow Plasma/Blood Separator Using Submicron Pillar Gap Structure,” conference of MicroTAS2009. For example, in the case of 0.67 μl/min of sample transfer rate, which is plasma transferring flow rate from the micro filter chip to nanowire biosensor, 10 μl of intermediate dead-volume consumes almost 15 minutes, which is almost half of the target total processing time of 30 minutes. Also the corresponding whole blood sample volume would be more than 300 μl just for filling up this dead-volume area, as described in T. G. Kang, et al, “A Continuous Flow Plasma/Blood Separator Using Submicron Pillar Gap Structure,” conference of MicroTAS2009. Thus, it may be beneficial to minimize the intermediate dead volume between the two modules, i.e. nanowire biosensor module and plasma separation module, in order to realize a microsystem having a faster processing capability with using ultra low sample volume. Further, it may also be desirable to minimize the dead volume of between the sample preparation module and the diagnostic module in order to maximizing the use of the blood sample.

In addition, for making early detection of these protein biomarkers to be ideal, it is essential to have a technology that can detect a panel of cardiac biomarkers with higher sensitivity e.g. less than 1 pg/ml (picograms per milliliter) detection limit, and faster response time e.g. within 30 minutes for total processing time in order for practitioners to provide timely treatments. To address higher sensitivity detection of proteins, the silicon nanowire biosensor technology has been built up (G.-J. Zhang, et al, “DNA Sensing by Silicon Nanowire: Charge Layer Distance Dependence,” Nano Letter, Vol.8 (2008) pp.1066-1070; G.-J. Zhang, et al, “Highly sensitive measurements of PNA-DNA hybridization using oxide-etched silicon nanowire biosensors,” Biosensors and Bioelectronics, Vol.23 (2008) pp.1701-1707; A. Agarwal, et al, “Nanowire sensor, naowire sensor array and method of fabricating the same” WO 2008/018834; G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009; and G. J. Zhang, et al, “Label-free direct detection of MiRNAs with silicon nanowire biosensors” Biosensors and Bioelectronics (2009), vol 24, pp. 2504) for electrical detection of biomolecules. The basic principle of nanowire biosensor for detection of biomarkers is as follows. The surfaces of the nanowires in a biosensor may be pre-treated to allow binding of biomarkers. For example, the biosensor may be pre-treated by immobilizing a certain kind of antibody on the surface of the nanowires in the biosensor. The antibody may be specific to bind a certain kind of biomarker. Upon detection of whether biomarkers exist in a tested sample, a change of nanowire resistance may indicate that bindings of the biomarkers with the pre-treated nanowires have taken place at the nanowire surface, and therefore it may be concluded that the biomarkers exist in the tested sample. The nanowire biosensors have been proved to have high sensitivity and specificity due to its large surface-to-volume ratio with label-free specific antibody-antigen reaction.

Thus, it is also desirable to make the diagnostic kit compatible with the high-end semiconductor fabrication technology such as nanowire fabrication process and sub-micron pillar gap structures described in T. G. Kang, et al, “A Continuous Flow Plasma/Blood Separator Using Submicron Pillar Gap Structure,” conference of MicroTAS2009.

### SUMMARY

Various embodiments provide an integrated micro device which includes a first microfluid device and a second microfluidic device and which may minimize the dead volume between the first and second microfluidic devices. The integrated micro device may be used as an integrated diagnostic kit, for example. The first microfluidic device may for example be a diagnostic module for detecting biomarkers and the second microfluidic device may for example be a sample preparation module. The integrated micro device may be compatible with various high-end semiconductor fabrication technology such as nanowire fabrication process and sub-micron pillar gap structures etc.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 illustrates a cross section of an integrated micro device according to one embodiment;

FIG. 2 (a) shows a cross section of an integrated micro device according to one exemplary embodiment;

FIG. 2 (b) shows a side view of an integrated micro device and images of both sides of the integrated micro device according to an exemplary embodiment;

FIG. 2 (c) illustrates the working principle of the integrated micro device;

FIG. 2 (d) illustrates photos of an integrated micro device according to one exemplary embodiment;

FIG. 3 illustrates a method for detecting biomarkers using the integrated micro device as described herein according to one embodiment;

FIG. 4 illustrates a method for manufacturing an integrated micro device according to one embodiment;

FIGS. 5 (a)-(n) show a manufacturing process of an integrated micro device as described herein according to one exemplary embodiment, wherein:

FIG. 5 (a) shows a SOI (semiconductor on insulator) structure;

FIG. 5 (b) shows that the first semiconducting layer is patterned such that a fin structure is formed from the first semiconducting layer;

FIG. 5 (c) shows that at least one nanowire is formed between the electrical interconnection portions of the fin structure from the fin portion of the fin structure;

FIG. 5 (d) shows that an impurity doping process is applied to the nanowire for the nanowire to be activated as a semiconductor transistor; FIG. 5 (e) shows that a second insulating layer is deposited on the first insulating later, the electrical interconnection portions of the fin structure and the at least one nanowire, and a portion of the second insulating layer is removed such that at least a portion of the electrical interconnection portions of the fin structure is exposed;

FIG. 5 (f) shows that a further impurity doping doping process is applied in order to make the exposed electrical interconnection portions more conductive;

FIG. 5 (g) shows that electric contacts are formed to connect to the electrical interconnection portions of the fin structure;

FIG. 5 (h) shows a passivation layer is formed on the second insulating layer, and the passivation layer is patterned such that at least a portion of electric contacts is exposed;

FIG. 5 (i) shows the passivation layer and the second insulating layer is further patterned;

FIG. 5 (j) shows that the bottom side of the substrate is polished;

FIG. 5 (k) shows that a pillar gap structured microfluidic device is formed over the bottom side of the substrate;

FIG. 5 (l) show a via hole through the structure shown in FIG. 5 (k) is formed by laser drilling, and a further layer of silicon dioxide is formed over the bottom side of the substrate and on the side wall of the via hole;

FIG. 5 (m) shows that a capping layer is bonded over the bottom side of the substrate;

FIG. 5 (n) shows that the nanowire is exposed;

FIG. 6 illustrates a cross section of an integrated micro device arrangement according to one embodiment;

FIG. 7 (a) shows an image wherein the pillar gap is around 2.6 to 2.9 μm;

FIG. 7 (b) shows an image wherein the pillar gap has been reduced to around 0.6 to 0.9 μm after a pillar gap reduction process;

FIG. 8 (a) illustrates binding of different biomarkers on the surface of the respective specific antibodies which are immobilized on nanowires of a biosensor; and

FIG. 8 (b) illustrates the resistance change for different nanowires upon the binding with the respective biomarkers.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. In this regard, directional terminology, such as “top”, “bottom”, “front”, “back”, “leading”, “trailing”, etc, is used with reference to the orientation of the Figure(s) being described. Because components of embodiments can be positioned in a number of different orientations, the directional terminology is used for purposes of illustration and is in no way limiting. Other embodiments may be utilized and structural, logical, and electrical changes may be made without departing from the scope of the invention. The various embodiments are not necessarily mutually exclusive, as some embodiments can be combined with one or more other embodiments to form new embodiments. The following detailed description therefore, is not to be taken in a limiting sense, and the scope of the present invention is defined by the appended claims.

The word “exemplary” is used herein to mean “serving as an example, instance, or illustration”. Any embodiment or design described herein as “exemplary” is not necessarily to be construed as preferred or advantageous over other embodiments or designs.

In one embodiment, an integrated micro device is provided. The integrated micro device may include a substrate, a first microfluidic device disposed over a first surface of the substrate, and a second microfluidic device disposed over a second surface of the substrate. The second surface of the substrate is opposite to the first surface of the substrate. The integrated micro device may further include at least one via hole through the substrate connecting the first microfluidic device and the second microfluidic device. Both the first microfluidic device and the second microfluidic device are monolithically integrated with the substrate.

In one embodiment, a method for detecting biomarkers using the integrated micro device as described herein is provided. The method may include supplying a sample containing plasma to the second microfluidic device. The method may further include guiding the sample through the second microfluidic device, thereby separating the plasma from the sample. The method may further include guiding the separated plasma through the at least one via hole connecting the second microfluidic device and the first microfluidic device. The method may further include collecting the separated plasma on the first microfluidic device for detecting the biomarkers.

In one embodiment, a method for manufacturing an integrated micro device is provided. The method may include monolithically forming a first microfluidic device over a first surface of a substrate. The method may further include polishing a second surface of the substrate, wherein the second surface of the substrate is opposite to the first surface of the substrate. The method may further include monolithically forming a second microfluidc device over the second surface of the substrate. The method may further include forming at least one via hole through the substrate such that the at least one via hole extends from the second surface of the substrate to the first surface of the substrate.

In one embodiment, an integrated micro device arrangement is provided. The integrated micro device arrangement may include an integrated micro device as described herein. The integrated micro device arrangement may further include a protection housing for protecting the at least one via hole connecting the first microfluidic device and the second microfluidic device of the integrated micro device. The protection housing may further include a bottom element configured to cover the second surface of the substrate of the integrated micro device as described herein. The protection housing may further include at least one gasket configured to seal the at least one via hole of the integrated micro device as described herein. The protection housing may further include at least one covering element configured to cover the gasket. The protection housing may further include at least one fixing element configured to fix the at least one covering element and the at least one gasket on the bottom element.

It should be noted that the embodiments describing the integrated micro device are also analogously valid for the corresponding method for detecting biomarkers using the integrated micro device as described herein, the method for manufacturing an integrated micro device, and the integrated micro device arrangement where applicable.

Various embodiments provide an integrated micro device. The integrated device may include a substrate, a first microfluidic device disposed over a first surface of the substrate, and a second microfluidic device disposed over a second surface of the substrate. The second surface of the substrate is opposite to the first surface of the substrate. The integrated micro device may further include at least one via hole (through-hole) through the substrate connecting the first microfluidic device and the second microfluidic device. The first microfluidic device may be monolithically integrated with the substrate. Further, the second microfluidic device may be monolithically integrated with the substrate.

In this context, the first and second microfluidic devices being monolithically integrated with the substrate means that both the first and the second microfluidic devices are uniformly formed over the substrate. Monolithical integration does not include the scenario wherein the first and the second microfluidic devices are formed separately and then bonded to the substrate.

In one embodiment, the first microfluidic device is configured as a biosensor arrangement. In a further embodiment, the biosensor arrangement includes at least one nanowire biosensor. Nanowire biosensors may be the one as described in G.-J. Zhang, et al, “DNA Sensing by Silicon Nanowire: Charge Layer Distance Dependence,” Nano Letter, Vol.8 (2008) pp.1066-1070; G.-J. Zhang, et al, “Highly sensitive measurements of PNA-DNA hybridization using oxide-etched silicon nanowire biosensors,” Biosensors and Bioelectronics, Vol.23 (2008) pp.1701-1707; A. Agarwal, et al, “Nanowire sensor, naowire sensor array and method of fabricating the same” WO 2008/018834; G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec 7-9, 2009; and G. J. Zhang, et al, “Label-free direct detection of MiRNAs with silicon nanowire biosensors” Biosensors and Bioelectronics (2009), vol 24, pp. 2504, for example. In one embodiment, the at least one nanowire biosensor is a silicon-nanowire biosensor. In one embodiment, at least a part of the at least one nanowire biosensor includes an exposed portion to receive a fluid, e.g. plasma.

For example, the surface of the nanowire in the biosensor may be pre-treated to allow binding with a specific biomarker. A test sample, e.g. plasma, may be in contact with the nanowire of the biosensor for detection of whether the specific biomarker exists in the test sample. If the test sample contains the biomarker, the biomarker may bind the surface of the nanowire and such binding may cause the resistance change of the nanowire. A detection of the change of resistance of the nanowire of the biosensor may indicate the existence of the biomarker in the test sample.

In one embodiment, the second microfluidic device is configured as a micro filter structure. The micro filter structure may be configured, for example, to filter blood cells from a blood sample.

In one embodiment, the second microfluidic device includes a pillar gap structured microfluidic device. The pillar gap structured microfluidic device may be the one as described in T. G. Kang, et al, “A Continuous Flow Plasma/Blood Separator Using Submicron Pillar Gap Structure,” conference of MicroTAS2009. The second microfluidic device may be configured to separate plasma from a blood sample. In one embodiment, the second microfluidic device is covered by a cap. In a further embodiment, the cap is made of glass.

In one embodiment, the at least one via hole includes a via hole microchannel.

In one embodiment, the integrated micro device further includes at least one inlet via hole for supplying a sample to the second microfluidic device.

In one embodiment, the integrated micro device further includes at least one electric terminal for connecting an electric wire with at least one microfluidic device. In a further embodiment, the at least one electric terminal is disposed over the first surface of the substrate. For example, the electric terminal may be used to test whether there is resistance change of the nanowire of the biosensor upon exposing a test sample with the nanowire.

In one embodiment, a thin film layer is arranged on the second microfluidic device. In a further embodiment, the thin film layer is arranged on the inner walls of the at least one via hole. The thin film layer may be made from silicon dioxide. For example, in case the second microfluidic device is a pillar gap structured microfluidic device similar as the one described in T. G. Kang, et al, “A Continuous Flow Plasma/Blood Separator Using Submicron Pillar Gap Structure,” conference of MicroTAS2009, the deposition of the thin film layer may be used to reduce the pillar gap in the pillar gap structured microfluidic device.

In one embodiment, the first microfluidic device is configured to detect biomarkers, which generally refer to substance used as an indicator of a biological state. The substance may be, but not limited to, biomolecule, nucleic acid, a polypeptide, a protein, a small organism molecule or inorganic molecule.

In one exemplary embodiment, the first microfluidic device is configured to detect protein biomarkers. In an exemplary application, the first microfluidic device may be configured to detect cardiac biomarkers, i.e. biomarkers as an indicator of cardiac disease. Cardiac protein biomakers include, for example, cTnT, CK-MM, and CK-MB.

In one embodiment, the second microfluidic device is made from silicon. In one embodiment, the gap width of the pillar structure in the second microfluidic device is smaller than 0.8 μm.

In one embodiment, the substrate comprises a chip. In a further embodiment, the chip is a silicon chip.

In one embodiment, the first microfluidic device is disposed over the top surface of the substrate. In one embodiment, the second microfluidic device is disposed below the bottom surface of the substrate.

In one embodiment, the substrate is made from silicon.

In one embodiment, a method for detecting biomarkers using the integrated micro device as described herein is provided. The method may include supplying a sample containing plasma to the second microfluidic device. The method may further include guiding the sample through the second microfluidic device, thereby separating the plasma from the sample. The method may further include guiding the separated plasma through the at least one via hole connecting the second microfluidic device and the first microfluidic device. The method may further include collecting the separated plasma on the first microfluidic device for detecting the biomarkers. In one exemplary embodiment, the biomarkers are protein biomarkers. In a further embodiment, the supplying of a sample containing plasma to the second microfluidic device includes injecting the sample through an inlet via hole and guiding the sample from the first surface of the substrate to the second surface of the substrate.

In one embodiment, a method for manufacturing an integrated micro device is provided. The method includes monolithically forming a first microfluidic device over a first surface of a substrate. In one embodiment, the method further includes polishing a second surface of the substrate, wherein the second surface of the substrate is opposite to the first surface of the substrate. In one embodiment, the method further includes monolithically forming a second microfluidc device over the second surface of the substrate. In one embodiment, the method further includes forming at least one via hole through the substrate such that the at least one via hole extends from the second surface of the substrate to the first surface of the substrate.

In one embodiment, the process of forming the first microfluidic device over the first surface of a substrate includes depositing a first insulating layer on the substrate. In one embodiment, the process of forming the first microfluidic device over the first surface of a substrate further includes depositing a first semiconducting layer on the first insulating layer. In one embodiment, the process of forming the first microfluidic device over the first surface of a substrate further includes patterning the first semiconducting layer such that a fin structure is formed. The fin structure may have a fin portion arranged between two electrical interconnection portions. In one embodiment, the process of forming the first microfluidic device over the first surface of a substrate further includes forming at least one nanowire from the fin portion between the electrical interconnection portions. In one embodiment, the process of forming the first microfluidic device over the first surface of a substrate further includes depositing a second insulating layer on the first insulating layer, the electrical interconnection portions, and on the at least one nanowire. In one embodiment, the process of forming the first microfluidic device over the first surface of a substrate further includes removing a portion of the second insulating layer such that at least a portion of the electrical interconnection portions is exposed. In one embodiment, the process of forming the first microfluidic device over the first surface of a substrate further includes forming electric contacts connected to the electrical interconnection portions. In one embodiment, the process of forming the first microfluidic device over the first surface of a substrate further includes forming a passivation layer on the second insulating layer. In one embodiment, the process of forming the first microfluidic device over the first surface of a substrate further includes patterning the passivation layer and the second insulating layer such that at least a portion of the at least one nanowire is exposed.

In a further embodiment, the forming of the second microfluidic device over the second surface of the substrate includes a lithography process, an etching process of the substrate, and deposition and etching process of an additional thin film layer to form pillar gap structures. The deposition and etching process of the additional thin film layer may be carried out by means of an anisotropic dry etching process. The width of the pillar gap structures may be reduced by repeating deposition and etching process of the additional thin film layer.

In one embodiment, the etching process of the substrate is carried out by means of a deep reactive ion etching process.

In one embodiment, the method for manufacturing an integrated micro device further includes forming a cover layer on the pillar gap structure over the second surface of the substrate. In a further embodiment, the cover layer is formed by means of anodic bonding. The cover layer may be formed as a glass wafer.

In one embodiment, at least a portion of the at least one nanowire is exposed by means of an exposing process. In a further embodiment, the exposing process is a wet exposing process. For example, the exposing process may be carried out using hydrogen fluoride (HF).

In one embodiment, the at least one via hole through the substrate connecting the first microfluidic device and the second microfluidic device is formed by a laser drilling process forming the at least one via hole from the first surface of the substrate to the second surface of the substrate.

In one embodiment, an integrated micro device arrangement is provided. The integrated micro device arrangement may include an integrated micro device as described herein. The integrated micro device arrangement may further include a protection housing for protecting the at least one via hole connecting the first microfluidic device and the second microfluidic device of the integrated micro device as described herein. The protection housing may include a bottom element configured to cover the second surface of the substrate of the integrated micro device as described herein. The protection housing may further include at least one gasket configured to seal the at least one via hole. The protection housing may further include at least one covering element configured to cover the gasket. The protection housing may further include at least one fixing element configured to fix the at least one covering element and the at least one gasket on the bottom element.

In one embodiment, the bottom element is a bottom plastic element.

In one embodiment, the fixing element includes a screw or a bolt.

In one embodiment, the surface of the at least on via hole of the integrated micro device as described herein is protected by the protection housing as described herein before performing the process of exposing at least a portion of the at least one nanowire.

FIG. 1 shows a cross section of an integrated micro device 100 in one embodiment.

The integrated micro device 100 includes a substrate 101. The integrated micro device 100 further includes a first microfluidic device 102 disposed over a first surface 110 of the substrate 101. The integrated micro device 100 further includes a second microfluidic device 103 disposed over a second surface 120 of the substrate 101. The second surface 120 of the substrate 101 is opposite to the first surface 110 of the substrate 101. The integrated micro device 100 further includes at least one via hole 104 through the substrate 101 connecting the first microfluidic device 102 and the second microfluidic device 103. The first microfluidic device 102 may be monolithically integrated with the substrate 101. The second microfluidic device 103 may be monolithically integrated with the substrate 101. In this context, the integrated micro device 100 as described herein may be referred to as a back-to-back integration structure.

In one embodiment, the first microfluidic device 102 is configured as a biosensor arrangement. In one embodiment, the biosensor arrangement includes at least one nanowire biosensor. In a further embodiment, the at least one nanowire biosensor is a silicon-nanowire biosensor.

In one embodiment, at least a part of the at least one nanowire biosensor comprises an exposed portion to receive a fluid.

In one embodiment, the second microfluidic device 103 is configured as a micro filter structure. In one embodiment, the second microfluidic device 103 includes a pillar gap structured microfluidic device. In one embodiment, the second microfluidic device 103 is configured to separate plasma from a blood sample.

In one embodiment, the second microfluidic device is made from silicon. In one embodiment, the gap width of the pillar structure is smaller than 0.8 μm.

In one embodiment, the second microfluidic device 103 is covered by a cap. For example, the cap may be made of glass.

In one embodiment, the at least one via hole 104 includes a via hole microchannel.

In one embodiment, the integrated micro device 100 further includes at least one inlet via hole (not shown) for supplying a sample to the second microfluidic device 103.

In one embodiment, the integrated micro device 100 further includes at least one electric terminal (not shown) for connecting an electric wire with at least one microfluidic device. In various embodiments, the at least one electric terminal is disposed over the first surface 110 of the substrate 101.

In one embodiment, a thin film layer (not shown) is arranged on the second microfluidc device 103. In various embodiments, the thin film layer is arranged on the inner walls of the at least one via hole 104. For example, the thin film layer may be made from silicon dioxide.

In an exemplary embodiment, the first microfluidic device 102 is configured to detect protein biomarkers. For example, the first microfluidic device 102 is configured to detect at least one of the biomarkers of cTnT, CM-MM, and CM-MB.

In one embodiment, the substrate 101 includes a chip or die. The chip may be a silicon chip.

In one embodiment, the first microfluidic device 102 is disposed over the top surface of the substrate 101, and the second microfluidic device is disposed below the bottom surface of the substrate 101.

In one embodiment, the substrate is made from silicon.

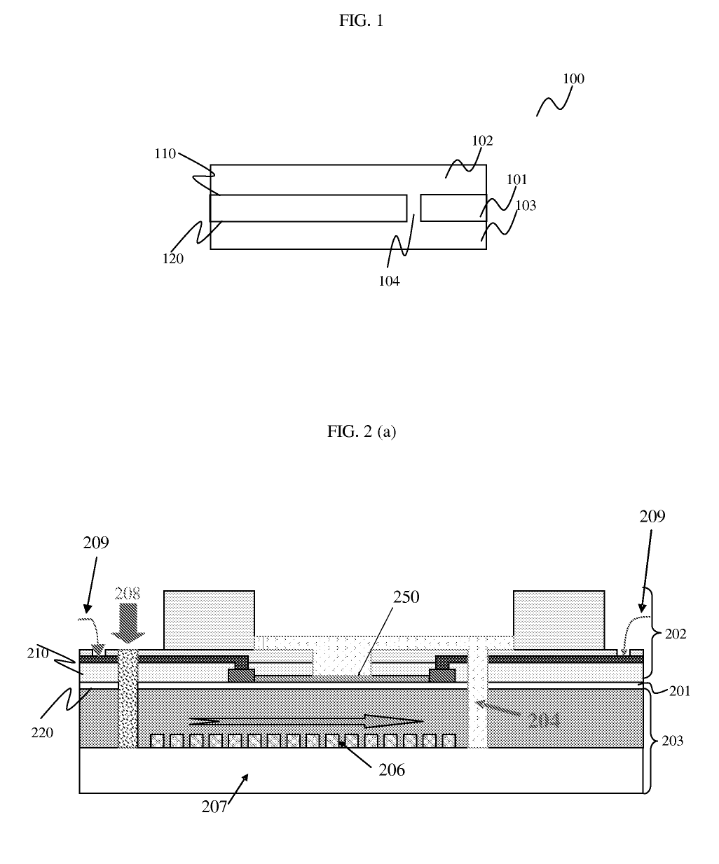

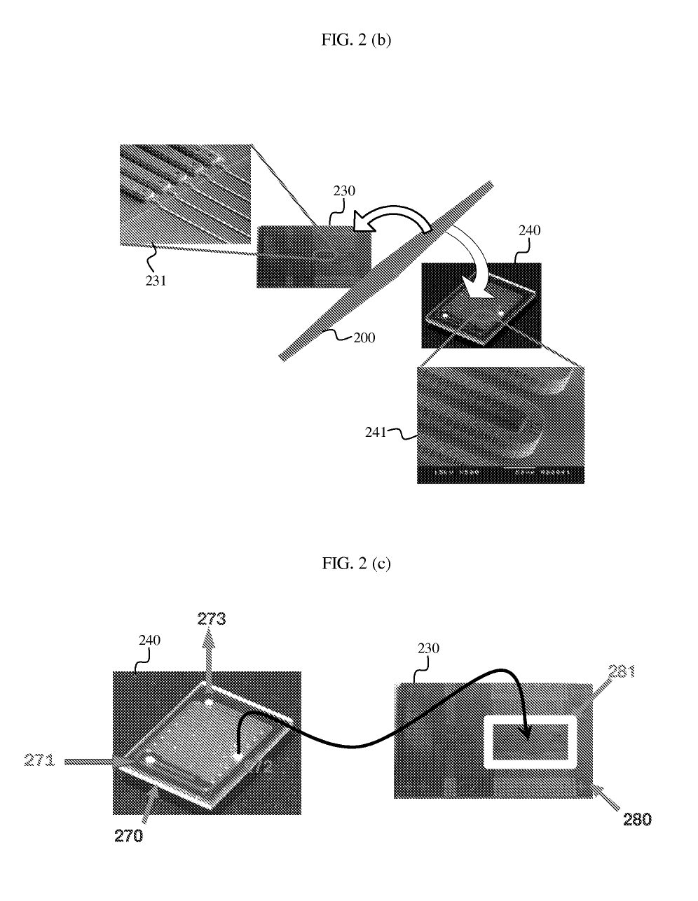

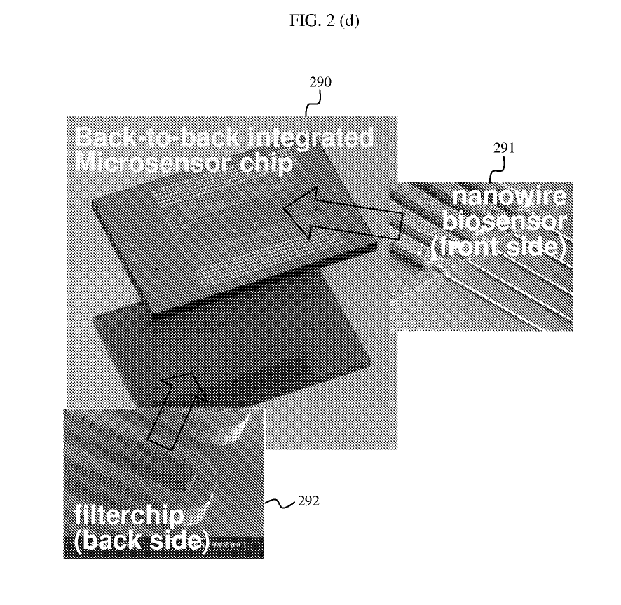

FIG. 2 (a) illustrates a cross section of an integrated micro device 200 according to one exemplary embodiment.

The integrated micro device 200 may include a substrate 201. The integrated micro device 200 may further include a first microfluidic device 202 disposed over a first surface 210 of the substrate 201 and a second microfluidice device 203 being disposed over a second surface 220 of the substrate 201. The second surface 220 is opposite to the first surface 210 of the substrate 201. Both the first microfluidic device 202 and the second microfluidic device 203 may be monolithically integrated with the substrate 201. The integrated micro device 200 further includes a via hole 204 through the substrate 201 connecting the first microfluidic device 202 and the second microfluidic device 203.

In this exemplary embodiment, the first microfluidic device 202 is configured as a biosensor arrangement which comprises at least one nanowire biosensor. An example of the nanowire biosensor may be the silicon-nanowire biosensor as described in G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009. The surface of the nanowire may be chemically modified to allow binding of specific biological probe molecules, e.g. at least one of cTnT, CM-MM, and CM-MB. The surface of the nanowires may be pre-treated by immobilizing antibodies on the surface of the nanowires. At least a part of the nanowire biosensor of the first microfluidic device 202 may include an exposed portion 250 to receive a fluid, e.g. plasma.

The second microfluidic device 203 is configured as a micro filter structure. For example, the second microfluidic device 203 may include a pillar gap structured microfluidic device 206. Such a pillar gap structure microfluidic device 206 may be, for example, the one as described in T. G. Kang, et al, “A Continuous Flow Plasma/Blood Separator Using Submicron Pillar Gap Structure,” conference of MicroTAS2009. The gap width of the pillar structure may be smaller than 0.8 μm. The second microfluidic device 203 may be made from silicon. The second microfluidic device 203 may be configured to separate plasma from a blood sample, for example.

The second microfluidic device may be covered by a cap 207. The cap 207 may be made from glass.

The at least one via hole 204 may include a via hole channel as shown in FIG. 2 (a).

The integrated micro device 200 may further include at least one inlet via hole 208 for supplying a sample to the second microfluidic device 203.

The integrated micro device 200 may further include at least one electric terminal 209 for connecting an electric wire with at least one of the first and second microfluidic devices 202 and 203. In the exemplary embodiment, the at least one electric terminal 209 is disposed over the first surface 210 of the substrate 201.

In one embodiment, a thin film layer (not shown) may be arranged on the second microfluidic device 203. The thin film may be arranged on the inner walls of the at least one via hole 204. For example, the thin film layer may be made from silicon dioxide.

The first microfluidic device 202 may be configured to detect biomarkers, e.g. protein biomarkers. For a further example, the first microfluidic device 202 may be configured to detect at least one of cTnT, CM-MM, and CM-MB.

The substrate 201 may include a chip or die. The chip may be a silicon chip.

In use, the first microfluidic device 202 may be disposed over the top surface of the substrate 201 and the second microfluidic device 203 may be disposed below the bottom surface of the substrate 202.

The substrate 201 may be made from silicon.

The working mechanism of the integrated micro device 200 is as follows.

For example, the integrated micro device 200 may be used to detect biomarkers, e.g. at least one of cTnT, CM-MM, and CM-MB, in a blood sample. The blood may be injected through the inlet via hole 208 into the second microfluidic device 203. The second microfluidic 203 may include a filtering structure such that plasma is separated from the blood sample. The blood may be guided through the pillar gap structured microfilter area for separation of plasma. The separated plasma may be fed into the first microfluidic device 202 through the via hole 204. The separated plasma may be collected in an open chamber for the biosensor detection. The first microfluidic device 202 may be configured as a biosensor, e.g. nanowire biosensor, and to detect whether biomarkers exist in the plasma. The detection of the existence of biomarkers may indicate that there is an injury to the heart muscle of the subject from which the blood sample is taken. In this structure, intermediate dead-volume between two functional devices (the first microfluidic device 202 and the second microfluidic device 203) is estimated to be around 0.05 μl only.

FIG. 2 (b) illustrates the side view of the integrated micro device 200 and the photos of the front view of the first microfluidic device 202 and photos of the front view of the second microfluidic device 203 according to an exemplary embodiment.

The image 230 shows the photo of a front view of the biosensor 201 as shown in FIG. 2 (a) in one exemplary embodiment. A further enlarged view of the circled area of the image 230 is shown in image 231. A more detailed description of the nanowire biosensor may be seen in G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009. The second microfluidic device 203 may be a filter chip. Image 240 shows a photo of the filter chip 203 in one exemplary embodiment. A further enlarged view of the circled area of the image 240 is shown in image 241. A more detailed description of the filter chip can be seen in T. G. Kang, et al, “A Continuous Flow Plasma/Blood Separator Using Submicron Pillar Gap Structure,” conference of MicroTAS2009.

As shown in FIG. 2 (b), integrated microdevice consists of a silicon nanowire biosensor 202 on the front side of the integrated micro device 200 and pillar gap structured micro filter structure 206 on the bottom side, which is covered by glass bottom-cap. These two microfluidic devices are connected through via-hole microchannel 204 in the integrated micro device 200. The silicon nanowire biosensor and pillar gap structured micro filter device have incompatible fabrication processes, and are thus not suitable to be fabricated on a same surface. Thus, it is desirable that the fabrication processes are separated at least into different surfaces. At the same time, in order to achieve faster total processing time and low sample consumption, the intermediate dead-volume between two devices needs to be minimized. The back-to-back integration structure provided herein provides a minimized dead volume between the two microfluidic devices.

FIG. 2 (c) further illustrates the working mechanism of the integrated micro device 200 as described herein according to an exemplary embodiment.

Image 240 shows a micro plasma separator 270 and image 230 shows a nanowire biosensor 280. The nanowire biosensor 280 and the micro plasma separator 270 are monolithically formed on different surfaces of a same substrate. The plasma separator 270 may be connected with the nanowire biosensor 280 via a via hole.

For example, a blood sample may be fed into a plasma separator 270 through an inlet 271. The plasma filter micro device 270 may be the same or similar as the one described in T. G. Kang, et al, “A Continuous Flow Plasma/Blood Separator Using Submicron Pillar Gap Structure,” conference of MicroTAS2009. The plasma filtered out may be fed through a plasma outlet 272 into the detection chamber 281 of the nanowire biosensor 280 through a via hole in the substrate. The waste-out of the blood sample may be collected at a waste-out end 273 of the plasma separator 270.

FIG. 2 (d) shows the photo 290 of a fabricated back-to-back integrated microchip according to one exemplary embodiment. The photograph 290 has been taken by using mirror and transparent spacer underneath of the integrated microchip. Sub-image 291 shows an enlarged image of the nanowire biosensor. Sub-image 292 shows an enlarged image of a portion of the filterchip.

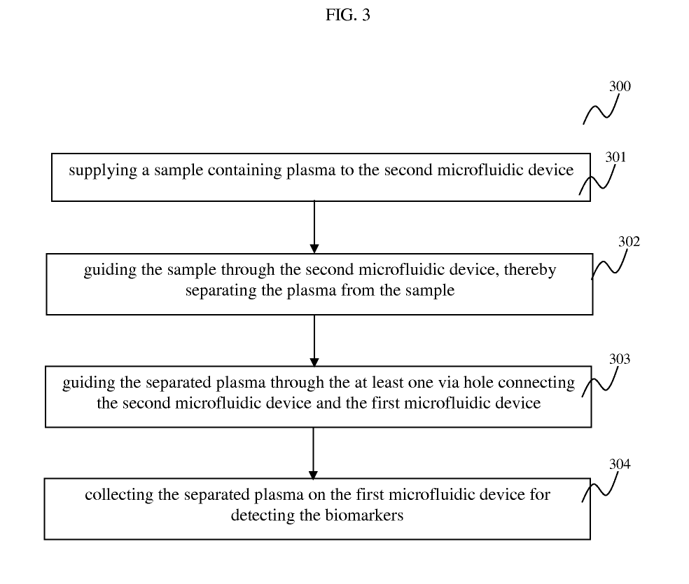

FIG. 3 illustrates a method 300 for detecting biomarkers using the integrated micro device as described herein according to one embodiment. The method may include 301 supplying a sample containing plasma to the second microfluidic device. The method may further include 302 guiding the sample through the second microfluidic device, thereby separating the plasma from the sample. The method may further include 303 guiding the separated plasma through the at least one via hole connecting the second microfluidic device and the first microfluidic device. The method may further include 304 collecting the separated plasma on the first microfluidic device for detecting the biomarkers.

In one embodiment, a sample containing plasma is supplied to the second microfluidic device by injecting the sample through an inlet via hole and the sample is guided from the first surface to the second surface of the substrate.

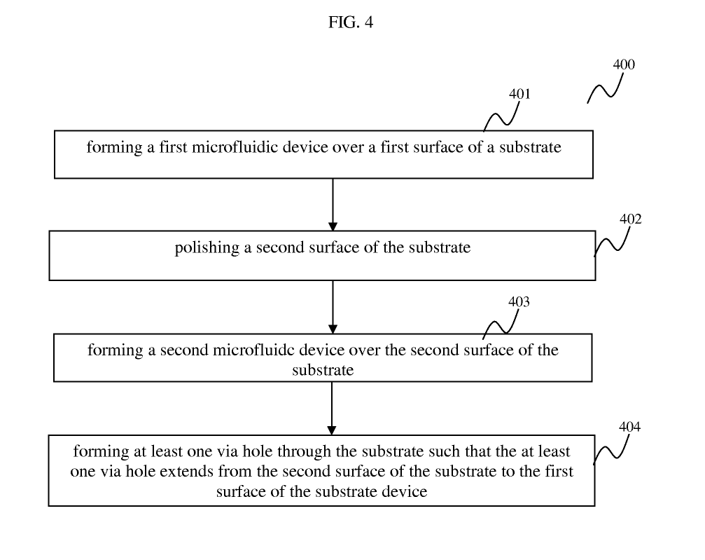

FIG. 4 illustrates a method 400 for manufacturing an integrated micro device.

In one embodiment, the method 400 for manufacturing an integrated micro device includes 401 monolithically forming a first microfluidic device over a first surface of a substrate. The method 400 may further include 402 polishing a second surface of the substrate. The second surface of the substrate is opposite to the first surface of the substrate. The method 400 may further include 403 monolithically forming a second microfluidic device over the second surface of the substrate. The method 400 may further include 404 forming at least one via hole through the substrate such that the at least one via hole extends from the second surface of the substrate to the first surface of the substrate device.

In one exemplary embodiment, the first microfluidic device formed over the first surface of the substrate may be a nanowire biosensor. According to this exemplary embodiment, the process of 401 forming the first microfluidic device over the first surface of a substrate may include depositing a first insulating layer on the substrate. The process of 401 forming the first microfluidic device over the first surface of a substrate may further include depositing a first semiconducting layer on the first insulating layer. The process of 401 forming the first microfluidic device over the first surface of a substrate may further include patterning the first semiconducting layer such that a fin structure is formed. The fin structure may include a fin portion arranged between two electrical interconnection portions. The process of 401 forming the first microfluidic device over the first surface of a substrate may further include forming at least one nanowire on the first insulating layer between the electrical interconnection portions. The process of 401 forming the first microfluidic device over the first surface of a substrate may further include depositing a second insulating layer on the first insulating layer, the electrical interconnection portions and on the at least one nanowire. The process of 401 forming the first microfluidic device over the first surface of a substrate may further include removing a portion of the second insulating layer such that at least a portion of the electrical interconnection portions is exposed. The process of 401 forming the first microfluidic device over the first surface of a substrate may further include forming electric contacts connected to the electrical interconnection portions. The process of 401 forming the first microfluidic device over the first surface of a substrate may further include forming a passivation layer on the second insulating layer. The process of 401 forming the first microfluidic device over the first surface of a substrate may further include patterning the passivation layer and the second insulating layer such that at least a portion of the at least one nanowire is exposed.

In a further embodiment, the process of 402 forming the second microfluidic device over the second surface of the substrate includes a lithography process, an etching process of the substrate, and deposition and etching process of an additional thin film layer to form pillar gap structures. The second microfluidic device may be a micro filter having a pillar gap structure according to one exemplary embodiment. The deposition and etching process of the additional thin film layer may be carried out by means of an anisotropic dry etching process. The width of the pillar gap structures may be reduced by repeating deposition and etching process of the additional thin film layer as described in T. G. Kang, et al, “A Continuous Flow Plasma/Blood Separator Using Submicron Pillar Gap Structure,” conference of MicroTAS2009. The etching process of the substrate may be carried out by means of a deep reactive ion etching process.

In one embodiment, the method 400 for manufacturing in integrated micro device further includes forming a cover layer on the pillar gap structure over the second surface of the substrate. The cover layer may be formed by means of anodic bonding. The cover layer may be formed as a glass wafer.

In one embodiment, at least a portion of the at least one nanowire is exposed by means of an exposing process. In a further embodiment, the exposing process is a wet exposing process. For example, the exposing process may be carried out using hydrogen fluoride.

In one embodiment, the at least one via hole through the substrate connecting the first microfluidic device and the second microfluidic device is formed by a laser drilling process forming the at least one via hole from the first surface of the substrate to the second surface of the substrate.

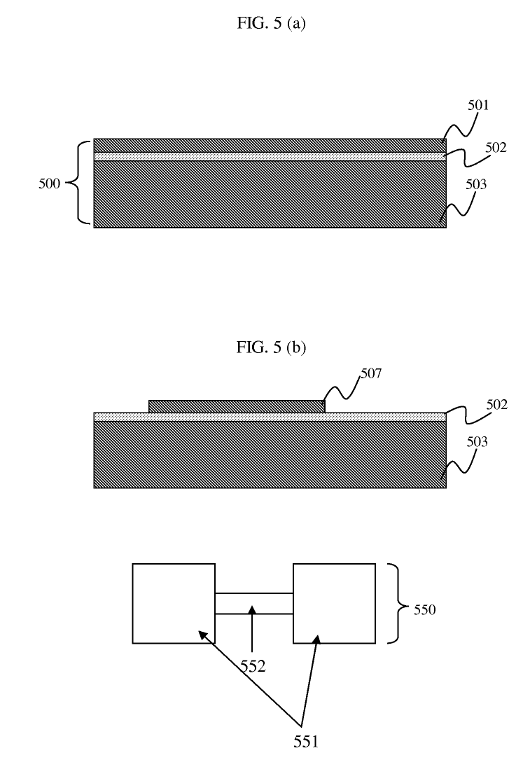

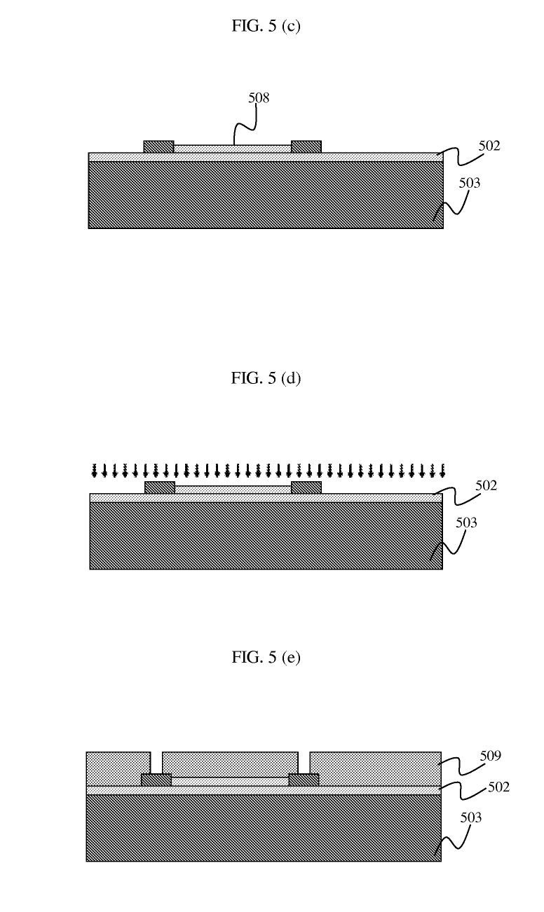

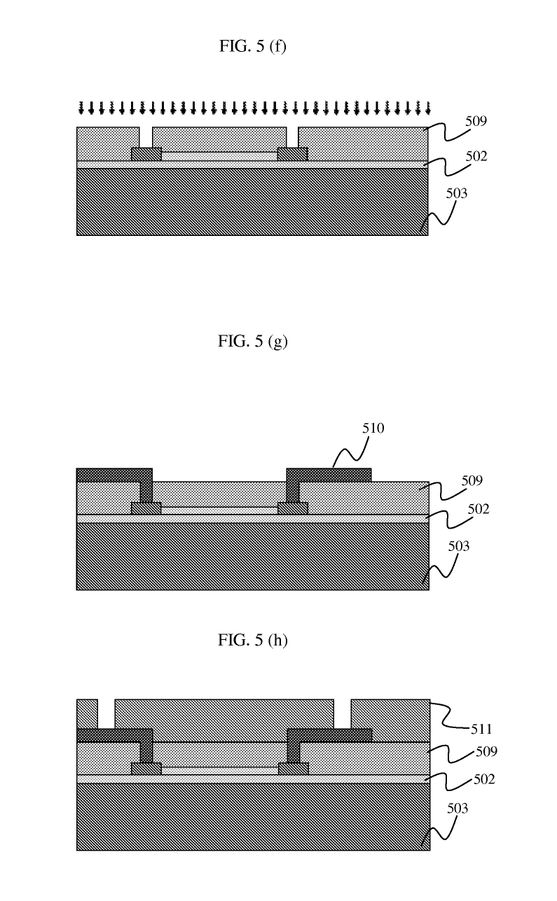

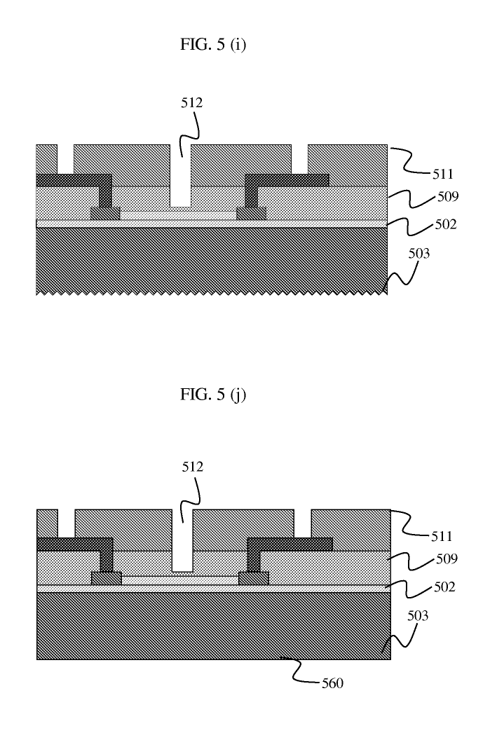

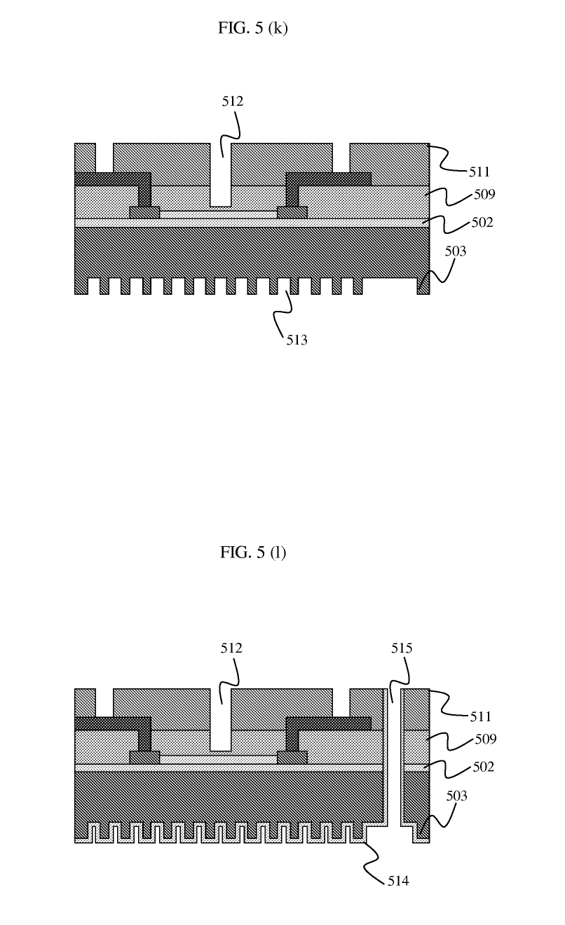

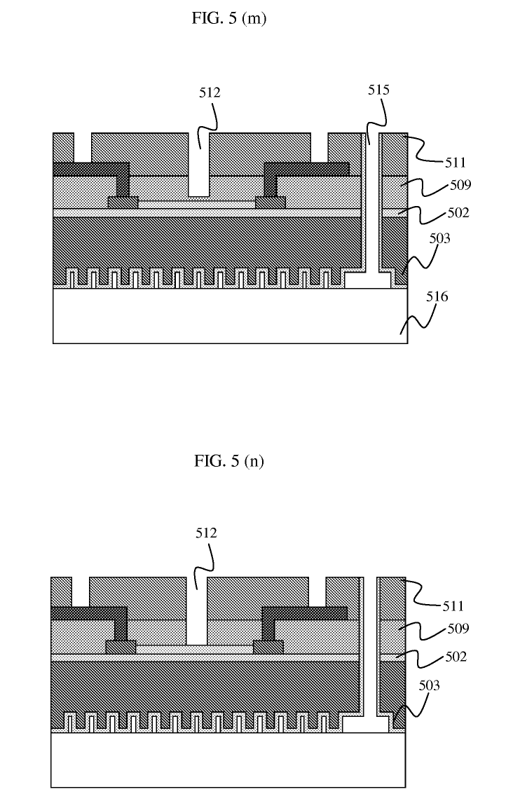

FIGS. 5 (a)-(n) illustrate a detailed fabrication process for forming an integrated micro device as described herein according to an exemplary embodiment. In this exemplary embodiment, the first microfluidic device is a nanowire biosensor similar as the one described in G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009. The second microfluidic device is a micro filter similar as the once described in T. G. Kang, et al, “A Continuous Flow Plasma/Blood Separator Using Submicron Pillar Gap Structure,” conference of MicroTAS2009. The first microfluidic device and the second microfluidic device are monolithically formed over a same substrate and on different sides of the substrate. It should be noted however that the first and the second microfluidic devices may be other type of microfluidic device depending on the specific application desired.

FIGS. 5 (a)-(i) and (n) show the fabrication process of forming a first microfluidic device over a substrate. FIGS. 5 (j)-(m) show the fabrication process of forming a second microfluidic device over the substrate. The first microfluidic device is a nanowire biosensor according to an exemplary embodiment and is monolithically formed on a substrate. The fabrication process of a nanowire biosensor has been illustrated in A. Agarwal, et al, “Nanowire sensor, naowire sensor array and method of fabricating the same” WO 2008/018834 and the process as illustrated in FIGS. 5 (a)-(i) is similar as the one described in A. Agarwal, et al, “Nanowire sensor, naowire sensor array and method of fabricating the same” WO 2008/018834. The second microfluidic device is a pillar gap structured filter device and the process illustrated in FIGS. 5 (j)-(m) is similar as the one described in T. G. Kang, et al, “A Continuous Flow Plasma/Blood Separator Using Submicron Pillar Gap Structure,” conference of MicroTAS2009. The fabrication process as illustrated in FIGS. 5 (a)-(n) is briefly described for illustration purpose.

FIG. 5 (a) shows a SOI (semiconductor on insulator) structure 500. The SOI structure 500 may be formed by depositing a first insulating layer 502, e.g. a buried oxide (BOX) layer, on a substrate 503 followed by depositing a first semiconductor layer 501 on the first insulating layer 502. The first semiconductor layer 501 is typically silicon but may be formed from any suitable semiconductor materials including, but not limited to, poly-silicon, gallium arsenide (GaAs), germanium or silicon-germanium (SiGe). The first semiconductor layer 501 may be initially doped with n-type dopants to render it n-type or p-type dopants to render it p-type. The substrate 503 may be formed from any suitable semiconductor materials including, but not limited to, silicon, sapphire, polycrystalline silicon (polysilicon), silicon dioxide (SiO₂) or silicon nitride (Si₃N₄). The BOX layer 502 is usually an insulating layer. The BOX layer 503 is typically silicon dioxide (SiO₂) based on tetraethylorthosilicate (TEOS), Silane (SiH₄) or thermal oxidation of Si, glass, silicon nitride (Si₃N₄) or silicon carbide having a thickness in the range of about 2 nanometers to about few micrometers, but is not limited to this.

FIG. 5 (b) shows that the first semiconductor layer 501 is patterned and part of the first semiconductor layer 501 is etched away such that a fm structure 507 is formed from the first semiconductor layer 501. 550 is a top view of the fin structure 507. The fin structure 507 may include a fin portion 552 arranged between two electrical interconnection portions 551. This process may be done by standard photolithography and etching techniques.

FIG. 5 (c) illustrates that a nanowire 508 is formed from the fin portion 552 of the structure 507. This may be achieved by a thermal oxidation process as described in A. Agarwal, et al, “Nanowire sensor, naowire sensor array and method of fabricating the same” WO 2008/018834.

FIG. 5 (d) illustrates that an implantation and activation process may be applied to dope the nanowire 508.

FIG. 5 (e) illustrates that a second insulating layer 509 is deposited on the first insulating layer 502, the electrical interconnection portions 551, and the at least one nanowire 508. After the deposition of the second insulating layer 509, a portion of the second insulating layer 509 may be removed such that at least a portion of the electrical interconnection portions 551 is exposed. This may be achieved by a standard photolithography technique.

FIG. 5 (f) illustrates that a contact doping process is further applied in order for providing higher conductivity to the electrical interconnection portions 551 of the fin structure.

FIG. 5 (g) illustrates forming electric contacts 510 connected to the electrical interconnection portions 551. The electric contacts 510 may be a conductive layer, portions of which being in contact with the electrical interconnection portions 551. The conductive layer 510 is usually a metal or a metal alloy. The metals can be but are not limited to aluminum, aluminum alloyed by Si, Copper (Cu) in various ratios, tantalum, tantalum nitride, titanium, titanium nitride, or a combination of these metals for example.

FIG. 5 (h) illustrates that a passivation layer 511 is formed on the second insulating layer 509 and the electric contacts 510. The passivation layer 511 may be formed from any suitable materials including, but not limited to, silicon dioxide (SiO₂) or silicon nitride (Si₃N₄). The passivation layer 511 may be further patterned such that a portion of the electric contacts 510 is exposed. This may be done through lithography and etching techniques commonly used in the art, such that later electrical potential may be applied to the metal lines through the pad opendings.

FIG. 5 (i) illustrates that the passivation layer 511 and the second insulating layer 509 are further patterned to form a channel 512 over the nanowire 508. The nanowire 508 remains to be covered by at least a portion of the second insulating layer 509. This may be done through lithography and dry etching techniques commonly used in the art.

FIG. 5 (j) shows that the backside 560 of the substrate 503 is polished for starting the backside fabrication process.

After achieving mirror surface on the backside 560 of the substrate 503 through the polishing process, photolithography process and deep Si RIE (reactive ion etching) process may be performed for forming the micropillar structures on the backside 560 of the substrate 503, which has been described in T. G. Kang, et al, “A Continuous Flow Plasma/Blood Separator Using Submicron Pillar Gap Structure,” conference of MicroTAS2009.

FIG. 5 (k) illustrates the formation of micropillar structures 513 on the backside of the substrate 503. Generally the etched initial pillar gap width can not be smaller than 0.8 μm, which is requested for red blood cell (RBC) filtration process.

FIG. 5 (l) shows that the pillar gap is reduced by repeating deposition of silicon dioxide (SiO₂) 514 and bare dry-etching without any masking layer, as described in T. G. Kang, et al, “A Continuous Flow Plasma/Blood Separator Using Submicron Pillar Gap Structure,” conference of MicroTAS2009. Through this gap reduction process, less than 0.8 μm pillar gap structure can be achieved. Further, a laser drilling process for forming the via hole microchannel 515 from the back side of the wafer to the front side may be applied.

FIG. 5 (m) shows that a glass wafer 516 is bonded over the backside for covering the micro pillar structure via anodic bonding.

FIG. 5 (n) shows that the nanowire 508 is released from the surrounding portion of the second insulating layer 509. An etching process such as wet etching may be used. The chemical etchant can be hydrofluoric acid (HF), for example.

For the final step for the fabrication process of nanowire release as described with reference to FIG. 5 (n), it is provided to block the via hole 515 from the chemicals such as hydrogen fluoride (HF). Once HF has penetrated to the via hole 515, it may damage the SiO2 surface of the microchannel 515. In one embodiment, an integrated micro device arrangement is provided which is able to block the silicon via hole 515 while HF SiO2 release as well as post surface chemical treatment for the nanowire surface. Hole-blocking is provided for nanowire surface functionalization by certain chemicals.

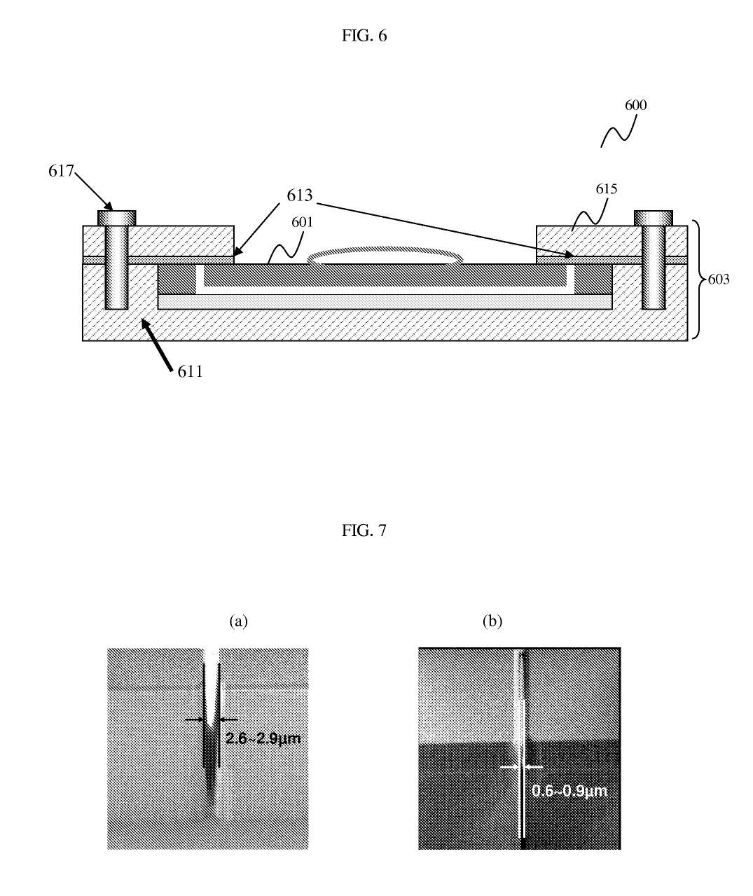

FIG. 6 shows an integrated micro device arrangement 600 according to one exemplary embodiment. The integrated micro device arrangement 600 may include an integrated micro device 601 as described herein. The integrated micro device arrangement 600 may further include a protection housing 603 for protecting the at least one via hole connecting the first microfluidic device and the second microfluidic device of the integrated micro device 601.

The protection housing 603 may include a bottom element 611 configured to cover the second surface of the substrate of the integrated micro device 601. The protection housing 603 may further include at least one gasket 613 configured to seal the at least one via hole of the integrated micro device 601. The protection housing 603 may further include at least one covering element 615 configured to cover the gasket 613. The protection housing 603 may further include at least one fixing element 617 configured to fix the at least one covering element 615 and the at least one gasket 613 on the bottom element 611.

The bottom element 611 may be a bottom plastic element. The fixing element 617 may include a screw or a bolt.

The surface of the at least one via hole of the integrated micro device may be protected by the protection housing 603 before performing the process of exposing at least a portion of the at least one nanowire as described with reference to FIG. 5 (n).

FIGS. 7 (a) and (b) show the pillar-gap reduction results. FIG. 7 (a) shows that before the pillar-gap reduction process by repeating SiO2 deposition and bare-etching, the pillar-gap was around 2.5 μm. FIG. 7 (b) shows that after three times repeating the process of deposition of silicon dioxide, the pillar gap has been reduced into less than 0.9 μm width. This has also been demonstrated in T. G. Kang, et al, “A Continuous Flow Plasma/Blood Separator Using Submicron Pillar Gap Structure,” conference of MicroTAS2009. It has been shown in G.-J. Zhang, et al, “DNA Sensing by Silicon Nanowire: Charge Layer Distance Dependence,” Nano Letter, Vol.8 (2008) pp.1066-1070; G.-J. Zhang, et al, “Highly sensitive measurements of PNA-DNA hybridization using oxide-etched silicon nanowire biosensors,” Biosensors and Bioelectronics, Vol.23 (2008) pp.1701-1707; A. Agarwal, et al, “Nanowire sensor, naowire sensor array and method of fabricating the same” WO 2008/018834; G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009; and G. J. Zhang, et al, “Label-free direct detection of MiRNAs with silicon nanowire biosensors” Biosensors and Bioelectronics (2009), vol 24, pp. 2504, that silicon nanowire array sensors can be used to carry out ultrasensitive, label-free, electrical detection of cardiac biomarkers in blood serum. The silicon nanowire array biosensor allows for real-time detection of cardiac biomarker in desalted serum and multiplexed detection of cardiac biomarkers in untreated and non-desalted blood serum.

The sensing mechanism of silicon nanowire biosensor as follows. The silicon nanowire surface is pretreated by immobilizing specific receptors for a corresponding specific biomarker onto the nanowire surface. For example, the biomarker may be a kind of protein and the receptor may be a kind of antibody which is capable of binding the corresponding specific protein. The binding may cause a change in charge density which induces a change in electric filed at the nanowire surface. Thus, a resistance change of the nanowire may indicate existence of the specific biomarker corresponding to the receptor. It is shown, in G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009, the specifity of the multiple antibodies-functionalized silicon nanowire sensors by selectively binding of various cardiac biomarkers to the antibodies and measuring the resistance change before and after the binding event. The binding only takes place on the silicon nanowire surface where they are specific, whereas no binding occurs on the clusters where the proteins are non-specific to the antibodies. It is also shown in G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009, that antibodies-functionalized silicon nanowire sensor shows a high sensitivity and is capable of multiplexed detecting proteins.

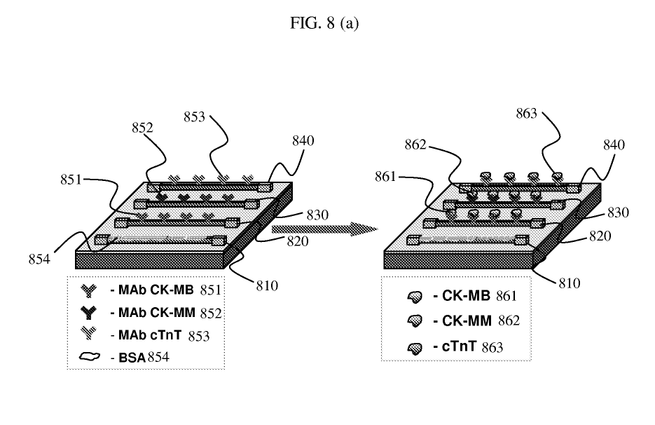

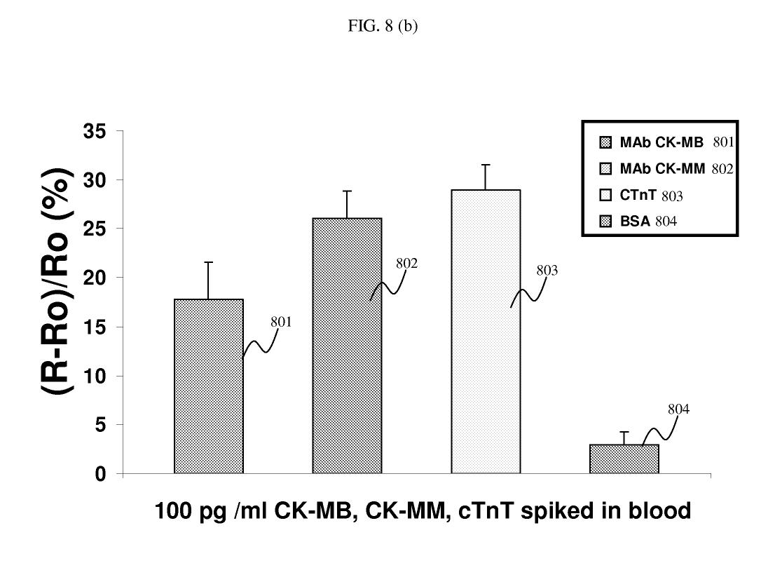

FIG. 8 (a) illustrates the working principle of the nanowire biosensor as described in G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009. In this illustration, four nanowires 810, 820, 830, 840 are formed over a substrate. Each nanowire may be pre-treated independently. For example, the nanowire 810 may be coated with bovine serum albumin (BSA) 854; antibodies MAb CK-MB 851 are immobilized on the surface of nanowire 820; antibodies MAb CK-MM 852 are immobilized on the surface of nanowire 830; and antibodies MAb cTnT 853 are immobilized on the surface of nanowire 840. The biosensor may selectively bind various cardiac biomarkers to the antibodies, and the binding of cardiac biomarkers to the surface-immobilized antibodies only takes place on the silicon nanowire surface where they are specific, whereas no binding occurs on the clusters where the proteins are non specific to the antibodies.

As shown in the right part of FIG. 8 (a), cardiac biomarkers CK-MB 861 are specifically bond to antibodies MAb CK-MB 851 immobilized on the nanowire 820; biomarkers CK-MM 862 are specifically bond to the antibodies MAb CK-MM 852 immobilized on the nanowire 830; and biomarkers cTnT 863 are specifically bond to antibodies MAb cTnT 853 immobilized on nanowire 840. No biomarkers of CK-MB, CK-MM, and cTnT bind to the BSA coated nanowire 810.

T. G. Kang, et al, “A Continuous Flow Plasma/Blood Separator Using Submicron Pillar Gap Structure,” conference of MicroTAS2009 shows a micro continuous flow plasma/blood separator, which is able to separate plasma from the blood sample by using submicron sized pillar gap structure. The working principle is basically the size-based exclusion of cells through cross-flow filtration. Only plasma can be allowed to pass through the submicron vertical pillars which are located tangential to the main flow path of the blood sample. The 0.6˜0.9 μm sized silicon pillar gap has been fabricated by repeating deposition and dry-etching processes of silicon dioxide (SiO₂) layer. The maximum filtration efficiency is measured as more than 99.9% with plasma collection rate of 0.67 μl/min at 12.5 μl/min input blood flow rate.

According to an exemplary embodiment, the silicon nanowire biosensor as described in G. J. Zhang, et al, “Highly Sensitive and Selective Label-Free Detection of Cardiac Biomarkers in Blood Serum with Silicon Nanowire Biosensors” 2009 IEEE International Electron Devices Meeting (IEDM), Baltimore, USA, Dec. 7-9, 2009, may be integrated with the plasma/blood separator as described in T. G. Kang, et al, “A Continuous Flow Plasma/Blood Separator Using Submicron Pillar Gap Structure,” conference of MicroTAS2009, to form an integrated micro device A. The integrated micro device A may have a cross section as shown in FIG. 2 (a), for example. That is, the silicon nanowire biosensor may be monolithically formed over a first surface of a substrate and the plasma/blood separator as described in T. G. Kang, et al, “A Continuous Flow Plasma/Blood Separator Using Submicron Pillar Gap Structure,” conference of MicroTAS2009, may be monolithically formed over a second surface of the substrate, the second surface being opposite to the first surface. The integrated micro device A may include a via hole connecting the silicon nanowire biosensor and the plasma/blood separator such that the plasma separated from the blood in the plasma/blood separator may be fed into the silicon nanowire biosensor through the via hole for further detection of biomarkers. Experiments have been carried out using such an integrated micro device A and results of resistance change are shown in FIG. 8 (b).

In the experiment, three different cardiac biomarker-linked antibodies involving MAb cTnT, MAb CK-MM, MAb CK-MB and BSA were separately spotted on the nanowires of the biosensor to allow selective multiplexed detection as shown in the left part of FIG. 8 (a). A blood sample comprising the biomarkers CK-MM, CK-MB, and cTnT is used in the experiment. Each of the biomarkers CK-MM, CK-MB, and cTnT has a concentration of 100 pg/ml. The blood sample is first injected to the plasma separator of the integrated micro device A. The plasma filtered out from the blood sample is then fed into the biosensor through the via hole of the integrated micro device A. FIG. 8 (b) shows the resistance change of different nanowires. 801 shows the resistance change of nanowire 820 on the surface of which antibodies MAb CK-MB 851 are immobilized. 802 shows the resistance change of nanowire 830 on the surface of which antibodies MAb CK-MM 852 are immobilized. 803 shows the resistance change of nanowire 840 on the surface of which antibodies MAb cTnT 853 are immobilized. 804 shows the resistance change of nanowire 810 on the surface of which BSA is coated. Thus, it can be seen that obvious change was obtained to each specific antibody spotted nanowire whereas negligible change was seen in case of binding of the individual protein to BSA.

Measurements has been conducted by using a probe station for verifying the integrated device performance.

Overall, the a back-to-back integrated structure for the integration of micro filter device together with silicon nanowire biosensor as well as its fabrication method for the integration have been provided. Two different microfluidic devices may be formed on back-to-back side of single semiconductor wafer, and connected through a via hole microchannel, as illustrated shown in FIG. 2 (a).

The integrated micro device as described herein is advantageous in that the intemediate dead volume can be minimized to around 0.05 μl, which can help to reduce total processing time by reducing sample transfer time from one to another and also reduce the blood sample volume. In addition, since both the first microfluidic device and the second microfluidic device are monolithically formed over a same substrate, there is no need for any additional substrate for interconnecting two different silicon-based microfluidic devices. Further, it is beneficial to form the first and the second microfluidic devices on different sides of the substrate especially when the fabrication process for the first microfluidic device is not compatible with that of the second microfluidic device. Furthermore, the integrated micro device as describe herein is more cost effective due to saving the foot-print area of silicon substrate and eliminating the additional substrate for the interconnection.

The structural of back-to-back integration of microfluidic devices such as silicon nanowire biosensor with pillar gap structured micro filter chip using silicon via-hole microchannel can be used for application to the integrated microsystem for point-of-care cardiac disease diagnostics tools. In addition to the previous high sensitive protein cardiac biomarker detection of using silicon nanowire biosensor, present back-to-back integration structure shows the capability of delivering faster diagnosis time as well as low sample consumption. The integrated fabrication method has also been provided for realization of the integrated microdevices having silicon nanowire biosensor on the front side of silicon wafer, pillar gap structured micro filter chip on the back side of the silicon wafer, and the silicon via-hole microchannel for the microfluidic interconnection. Various embodiments may show a potential for the application to the cardiac disease point-of-care diagnostics tool with high sensitivity, faster processing time as well as low blood-sample consumption. The integrated micro device as described herein can be used to disease diagnostics based on the protein biomarker detection, for example. The integrated micro device as described herein also shows the potential to application to an integrated microsystem for disease diagnostics, and it may enable detection and diagnostics in an early stage and benefit saving lives for human being.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. An integrated micro device, comprising:
a substrate;
a first microfluidic device disposed over a first surface of the substrate;
a second microfluidic device disposed over a second surface of the substrate, the second surface of the substrate being opposite to the first surface of the substrate; and
at least one via hole through the substrate connecting the first microfluidic device and the second microfluidic device;
wherein the first microfluidic device is monolithically integrated with the substrate; and
wherein the second microfluidic device is monolithically integrated with the substrate.

2. The integrated micro device according to claim 1,
wherein the first microfluidic device is configured as a biosensor arrangement.

3. The integrated micro device according to claim 1,
wherein the second microfluidic device is configured as a micro filter structure.

4. The integrated micro device according to claim 2,
wherein the biosensor arrangement comprises at least one nanowire biosensor.

5. The integrated micro device according to claim 4,
wherein the at least one nanowire biosensor is a silicon-nanowire biosensor.

6. The integrated micro device according to claim 4,
wherein at least a part of the at least one nanowire biosensor comprises an exposed portion to receive a fluid.

7. The integrated micro device according to claim 1,
wherein the second microfluidic device comprises a pillar gap structured microfluidic device.

8. The integrated micro device according to claim 1,
wherein the second microfluidic device is configured to separate plasma from a blood sample.

9. The integrated micro device according to claim 1,
wherein the second microfluidic device is covered by a cap.

10. The integrated micro device according to claim 1,
wherein the at least one via hole comprises a via hole microchannel.

11. The integrated micro device according to claim 1, further comprising:
at least one inlet via hole for supplying a sample to the second microfluidic device.

12. The integrated micro device according to claim 4, further comprising:
at least one electric terminal for connecting an electric wire with at least one microfluidic device.

13. The integrated micro device according to claim 1,
wherein a thin film layer is arranged on the second microfluidic device.

14. The integrated micro device according to claim 13,
wherein the thin film layer is arranged on the inner walls of the at least one via hole.

15. The integrated micro device according to claim 13,
wherein the thin film layer is made from silicon dioxide.

16. The integrated micro device according to claim 2,
wherein the first microfluidic device is configured to detect biomarkers.

17. The integrated micro device according to claim 16,
wherein the first microfluidic device is configured to detect troponins or creatinine kinases.

18. The integrated micro device according to claim 1,
wherein the second microfluidic device is made from silicon.

19. The integrated micro device according to claim 7,
wherein the gap width of the pillar structure is smaller than 0.8 μm.

20. The integrated micro device according to claim 1,
wherein the substrate comprises a chip.

21. The integrated micro device according to claim 20,
wherein the chip is a silicon chip.

22. The integrated micro device according to claim 1,
wherein the first microfluidic device is disposed over the top surface of the substrate; and
wherein the second microfluidic device is disposed below the bottom surface of the substrate.

23. A method for detecting biomarkers using the integrated micro device according to claim 1, the method comprising the steps of:
supplying a sample containing plasma to the second microfluidic device;
guiding the sample through the second microfluidic device, thereby separating the plasma from the sample;
guiding the separated plasma through the at least one via hole connecting the second microfluidic device and the first microfluidic device; and
collecting the separated plasma on the first microfluidic device for detecting the biomarkers.

24. A method for manufacturing an integrated micro device, the method comprising:
monolithically forming a first microfluidic device over a first surface of a substrate;
polishing a second surface of the substrate, wherein the second surface of the substrate is opposite to the first surface of the substrate;
monolithically forming a second microfluidc device over the second surface of the substrate;
forming at least one via hole through the substrate such that the at least one via hole extends from the second surface of the substrate to the first surface of the substrate.

25. An integrated micro device arrangement, comprising:
an integrated micro device according to claim 1; and
a protection housing for protecting the at least one via hole connecting the first microfluidic device and the second microfluidic device, the protection housing comprising:
a bottom element configured to cover the second surface of the substrate;
at least one gasket configured to seal the at least one via hole;
at least one covering element configured to cover the gasket;
at least one fixing element configured to fix the at least one covering element and the at least one gasket on the bottom element.
